(19) United States
(12) Patent Application Publication
(10) Pub. No.: US 2010/0104513 A1
(43) Pub. Date: Apr. 29, 2010

(54) METHOD AND SYSTEM FOR DYE ASSESSMENT

(75) Inventors: Jens Rittscher, Ballston Lake, NY (US); Umesha Perdoor Srinivas Adiga, Clifton Park, NY (US); Kenneth Michael Fish, Clifton Park, NY (US); Anup Sood, Clifton Park, NY (US); Kathleen Bove, Ballston Lake, NY (US); Evelina Roxana Loghin, Rexford, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: General Electric Company, Schenectady, NY (US)

(21) Appl. No.: 12/259,944

(22) Filed: Oct. 28, 2008

(51) Int. Cl. A61K 49/00; G06K 9/00

(52) U.S. Cl. 424/9.1; 382/128

(57) ABSTRACT

The present disclosure generally relates to systems and methods for identifying the boundaries of tumors and assessing quantitatively the ability of dyes to highlight a tumor's boundary. In accordance with these methods and systems, images are taken of subjects administered agents labeled with dyes. After accessing the images, tumors are selected and routines employed to both identify the boundaries of the tumors, as well as, to quantify various aspects of the tumor boundaries. From these quantifiable descriptors the performances of the various dyes to highlight the boundaries of tumors are evaluated.

### BACKGROUND

The invention relates generally to the field of tumor visualization. More particularly, the invention relates to the evaluation and selection of dyes for tumor visualization.

In operative procedures to remove tumors, the surgeon's ultimate goal consists of removing all of the cancerous tissue while sparing as much of the normal tissue as possible. A surgeon must make a visual assessment of the outer boundary of the tumor and then try to completely resect the tumor. A successful resection of the whole tumor generally results in a greater 5-year survival rate for patients than a partial resection. Various imaging techniques may be used preoperatively or intraoperatively in order to determine the extent of the tumor. However, these images may fail to identify the outer layer of the tumor. Thus, after resection of the tumor some tumor cells may remain. The continued presence of such tumor cells may be problematic to the extent that residual tumor cells can lead to a local recurrence and, thus, properly identifying and removing the tumor boundary is a key focus in surgery to remove a tumor.

As one might expect, factors that impact the likelihood of local recurrence include the skill of the surgeon performing the tumor resection and the information available to the surgeon. In particular, as suggested above, one reason why surgical treatment may fail in the early stages of cancer is because the entire tumor may not be removed (i.e., lack of clear margins). At present, the surgeon typically relies on visual inspection and palpitation during tumor resection. However it is often difficult to distinguish cancer tissue from normal tissue by sight and/or by touch.

Therefore, information that may be used to delineate the tumor boundary intra-operatively may improve the effectiveness of resection procedures and thereby diminish the probability of local tumor recurrence. Given the importance of correctly identifying the boundaries of tumors, there is a need to develop tools to help recognize and highlight the tumor boundary in a variety of clinical contexts.

### BRIEF DESCRIPTION

The present disclosure relates to the automatic identification of tumor boundaries with in image or images and the quantification of characteristics of these boundaries. In one embodiment, user input is provided to locate a dye-stained tumor in an image and, based upon this input, automated routines are employed to identify the boundary of the tumor. Characteristics of the boundary (such as measures related to average intensity, variance, contrast, or breaks in the boundary) may then be automatically measured and quantified and used as a basis for comparing the performance of the dye to other dyes or for comparing the performance of the same dye in different clinical contexts. In some embodiments, an intensity level standardization may be performed to standardize the intensity levels in each image so that the comparison of boundary characteristics between images is more meaningful.

In one embodiment, a method is provided that includes the act of accessing an image of a subject. The subject is administered an agent labeled with a dye prior to generation of the image. A tumor labeled with the dye is selected from the image. A first routine is employed to detect some or all of the boundary of the tumor. A second routine is employed to measure one or more characteristics of the boundary.

In another embodiment, a method for selecting dyes is provided that includes the act of accessing a plurality of images of tumors. The tumors are each stained with a respective image-enhancing dye of a plurality of dyes prior to imaging. The plurality of images are processed to identify the respective tumor boundaries within each image. One or more routines are employed to calculate one or more quantitative characteristics of each tumor boundary. One or more of the plurality of dyes are selected based on the one or more quantitative characteristics.

In another embodiment, a method for processing infrared image data to identify a tumor's boundary is provided. The method includes the act of administering an agent labeled with a fluorescent dye to a subject. An infrared image of the subject is generated and a tumor is selected from the image. A first computer-implemented algorithm is executed to identify the tumor's boundary. A second computer-implemented algorithm is executed to generate one or more quantitative characteristics of the tumor boundary. The one or more quantitative characteristics are reviewed to assess the performance of the fluorescent dye.

In another embodiment, a method is provided that includes the act of receiving an input indicative of the location of a dye-enhanced tumor in an image. A first routine configured to determine the boundary of the tumor in the image is executed. A second routine configured to calculate one or more quantitative characteristics of the boundary of the tumor is executed. The one or more quantitative characteristics are stored or displayed.

In yet another embodiment, a system is provided. The system includes a display capable of displaying an image of a dye-enhanced tumor and an input device configured to receive an operator input indicative of the location of the dye-enhanced tumor in the image. the system also includes a storage or memory device storing routines for determine the boundary of the dye-enhanced tumor and for calculating one or more quantitative characteristics of the boundary. In addition, the system includes a processor configured to receiving the operator input, to execute the routines stored in the storage or memory device in view of the operator input, and to display the one or more quantitative characteristics on the display.

### DRAWINGS

These and other features, aspects, and advantages of the present invention will become better understood when the following detailed description is read with reference to the accompanying drawings in which like characters represent like parts throughout the drawings, wherein:

FIG. 1 is a flow chart depicting acts for characterizing tumor boundaries according to one aspect of the present disclosure;

FIG. 2 is a screenshot illustrating the selection of a tumor and identification of the tumor's boundary according to one aspect of the present disclosure

FIG. 3 is a screenshot illustrating the identification of a tumor's boundary and display of quantitative characteristics associated with the boundary according to one aspect of the present disclosure;

FIG. 4 is a flow chart acts for selecting dyes according to one aspect of the present disclosure; and

FIG. 5 is a schematic representation of a processor-based system for executing routines used in implementing aspects of the present disclosure.

### DETAILED DESCRIPTION

As used herein, the term dye or dyes includes (but is not limited to) organic or inorganic fluorophores, fluorescent nanoparticles, fluorescent beads as well as their derivatives and conjugates to other molecules/vectors. Further, a vector is a vehicle that is used to transport the dye to one or more desired locations and may be targeted actively or passively. The use of dyes such as these to aid in visualizing certain medical phenomena is established. For example, certain dyes may be utilized to differentially highlight certain tissue types or structures, such as tumors. Such dyes may take advantage of particular properties of the tissues being highlighted.

Various approaches exist for developing agent, such as dyes, to highlight tumor tissue. For example, one approach, known as active targeting, targets tumor specific molecular targets, e.g. receptors, proteases, etc. (active targeting). Another approach, known as passive targeting, targets tumor morphology, e.g., leaky vasculature. Agents, i.e., dyes, developed using these types of approaches may be used to differentially highlight tumor structures. Such dyes may then be utilized in invasive procedures to allow a surgeon to visualize the extent of the tumor and to better facilitate removal of all tumor cells.

However, different types of tumors, subjects, or procedures may benefit from different dyes, i.e., different circumstances may call for different dyes. The number of potential suitable dyes, however, is vast and present techniques utilize subjective assessment which is qualitative in nature to screen candidate dyes or use manual procedures to highlight areas of interest before quantification. The latter approach is also subjective as a person visually identifies area of interest for quantification. In addition, manual identification is also laborious and time consuming. Such subjective assessments are generally unsuitable for screening large numbers of candidate dyes and, further, do not facilitate making meaningful comparisons between the candidates dyes.

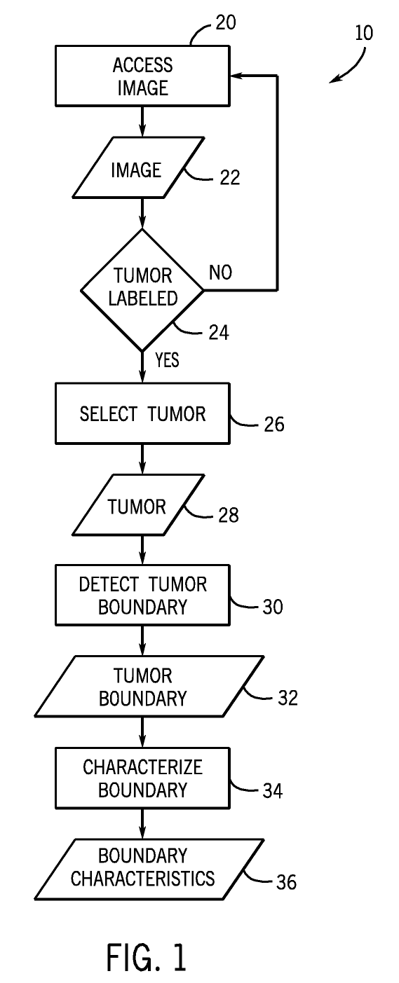

In addressing this issue, therefore, it may be desirable to provide a more quantitative assessment and to utilize automation where possible. With this in mind, reference is now made to FIG. 1 which depicts certain acts of one embodiment of such a method 10. In the embodiment of the technique described in FIG. 1, an operator accesses (block 20) an image 22 from a subject, such as a lab rat, administered a visualization agent, such as a suitable tumor specific dye, prior to the generation of the image 22. For example, the subject may be injected with a compound or solution that includes a fluorescing dye that preferentially accumulates in angiogenic tissues, such as tumors. The subject may then be surgically opened to expose the likely tumor location and one or more images 22 generated of the site. In one embodiment, an infrared (IR) imager (such as a system suitable for near infrared (NIR) fluorescent intra-operative imaging) is used to obtain one or more images of the dye-stained tumor. Thus, the images 22 accessed by the operator may be IR, NIR, or other suitable images of one or more dye-stained tumors. Certain wavelengths, such as NIR wavelengths, may be useful where less autofluorescence of standard tissues is desired.

In one embodiment, an operator may visually inspect the image 22 to determine (block 24) if the image 22 depicts a tumor that is suitably or sufficiently labeled with dye. In such an embodiment, the operator may consider factors such as whether the dye highlights only the boundary of the tumor (i.e., the tumor margin), whether the dye extends beyond the tumor or tumor boundary to an unacceptable degree, as well as, other aspects of proper labeling. If the operator decides the depicted tumor is not suitably labeled, the operator may access a different image 22. If the operator decides that the depicted tumor is suitably labeled, the operator may proceed to process the image 22.

Once a suitable image 22 is identified, the operator may select (block 26) the dye-labeled tumor 28 in the displayed image 22. For example, the operator may employ a mouse, touchpad, touchscreen, or other suitable point-and-select interface to select the tumor 28, such as by “clicking” on the perceived center of the tumor using a mouse or other suitable selection input device. In other embodiments, selection of the tumor 28 may be automated or semi-automated, such as by employing thresholding or other algorithms that identify concentrations of the dye over a certain limit within the image 22. In such embodiments, a tumor 28 may be tentatively identified based on the thresholding algorithms alone or potential tumors may be identified on the image 22 by the algorithm for further review and selection by an operator.

Once a tumor 28 is identified, one or more automated routines may be employed to detect (block 30) the boundary 32 of the tumor 28. The routine 18 may detect the entire boundary 32 of the tumor 28 or only a portion of the boundary 32, depending on the extent the dye highlights the boundary 32 of the tumor 28. In one embodiment, this routine, as well as others discussed herein, is implemented using the IDL language and can be distributed using the IDL virtual machine.

In one embodiment, another automated routine may be employed to measure (block 34) one or more quantitative characteristics 36 of the boundary 32. Examples of such boundary characteristics, as discussed in greater detail below, include average intensity, pixel intensity variance, number and relative length of boundary discontinuities, brightness ratio, average contrast, clearance rate, and so forth. The characteristics 36 of the boundary 32 may be reviewed or evaluated by an operator to evaluate or compare the efficacy of the dye in staining the tumor 28. In addition, the characteristics 36 may be stored for later review or comparison. As will be appreciated, some of the steps depicted in the flow chart of FIG. 1 may be optional in various embodiments.

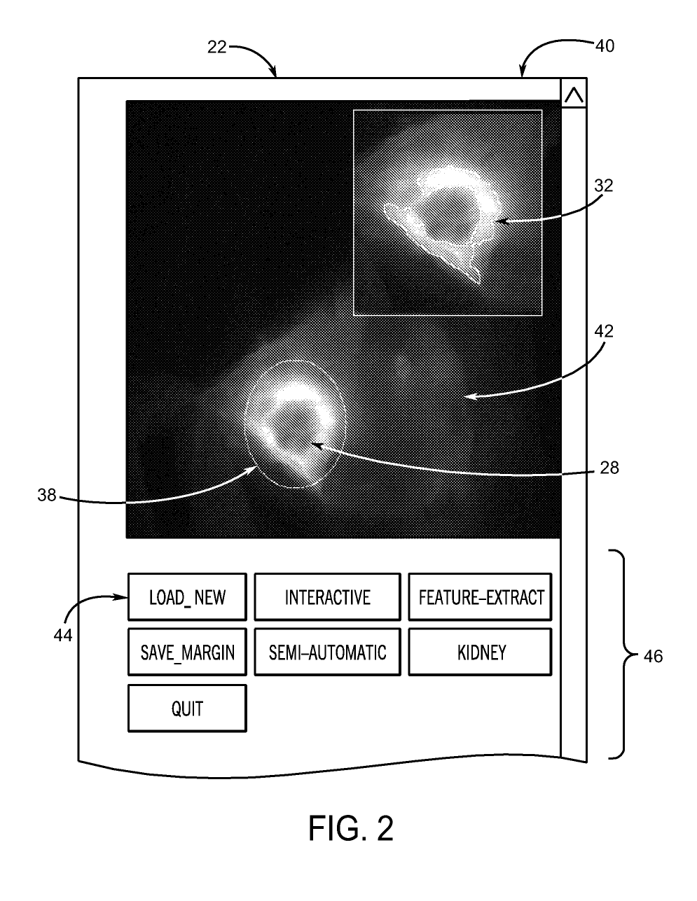

With the foregoing general discussion the following example is provided by way of illustration. Turning now to FIG. 2, a screenshot 40 displaying an infrared image 22 is depicted. In this example, infrared image 22 depicts a tumor 28 within an organ 42, such as the skin, kidney, spleen, liver, prostate, and so forth. If the image 22 is deemed to be unsuitable, such as due to insufficient staining of the tumor 28, an operator may load a new image, such as using the “LOAD NEW” button 44 of the user input interface 46. If, however, the image 22 is deemed suitable, the operator may select the tumor 28 from the image 22, such as using a mouse, touchscreen, or other point-and-select device to select the center of the perceived tumor 28. In one embodiment, the tumor selection process may be facilitated by the display of a circle 38 or other selection area that may be centered around a point selected by the operator or which may be moved by the operator to encompass the area deemed to show the tumor 28. Alternatively, as noted above, automatic or semi-automatic processes may be employed, in lieu of operator input, to select the tumor 28 within the image 22.

In certain embodiments, the image 22 may be processed prior to tumor selection and/or identification of the tumor boundary. For example, in one embodiment, the image 22 may be enhanced, such as by implementation of anisotropic smoothing and/or other pre-processing filters. In addition, in certain embodiments the image 22 may undergo contrast stretching and/or multi-stage binarization.

Once the tumor 28 is selected a computer-executed algorithm may automatically identify the tumor boundary 32. In one embodiment, the tumor boundary 32 may be identified utilizing an intensity threshold. Pixels having an intensity greater than a set or threshold value may be determined to correspond to tumor tissue. In turn, those pixels determined to correspond to tumor tissue that have intensity values greater than a neighboring pixel in at least one direction may be determined to correspond to the boundary 32 of the tumor 28. That is, those pixels which are stained (e.g., fluorescing) but which are adjacent to at least one other pixel that is not stained (e.g., non-fluorescing) above a certain threshold may be identified as corresponding to the boundary 32 of the tumor 28.

In one embodiment, upon determination of the tumor boundary 32, the circle 38 used to highlight the region having the tumor 28 may be warped to highlight the identified tumor boundary 32, as depicted in the inset to FIG. 2. For example, in one implementation, the tumor boundary 32 may be fitted using a generally annular or toroidal model, i.e., a doughnut or ring shaped model, which may be derived using the circle 38 used to highlight the region. Such an annular model may be suitable in implementations where the dye is generally expected to only highlight the peripheral region of the tumor, such as due to cellular death at the center of the tumor.

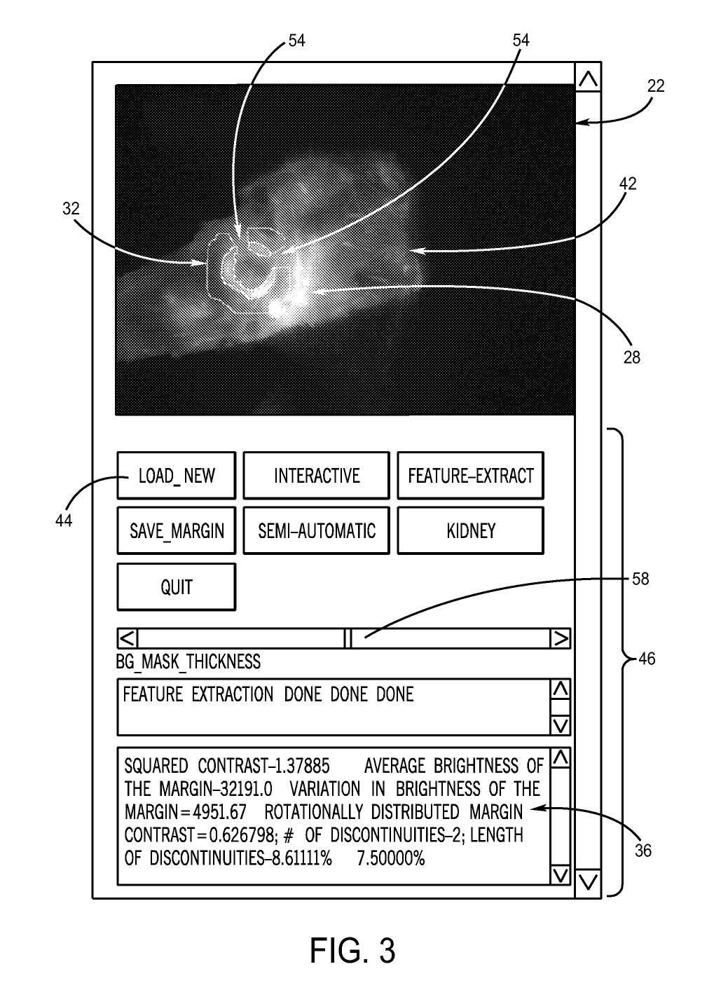

Turning now to the screenshot depicted in FIG. 3, once the tumor boundary 32 is identified, a computer-executed algorithm may be employed to quantify one or more aspects of the tumor boundary 32, such as by generating one or more boundary characteristics 36, such as quantitative descriptors, of the tumor boundary 32. An operator may review the boundary characteristics, such as to assess the performance of the fluorescent dye used in generating the specific image 22 under review, and/or the boundary characteristics may be stored for subsequent review or comparison.

In one embodiment, the algorithm employed may generate quantitative boundary characteristics 36 of one or more aspects of the tumor boundary 32. For example, in one embodiment, a quantitative descriptor of the average brightness of the tumor boundary 32 may be measured by averaging the intensity values of those pixels determined to correspond to the tumor boundary 32. Similarly, other measures of central tendency such as median and mode values, may be calculated based on the intensity values of those pixels determined to correspond to the tumor boundary 32. These descriptors may then be stored or displayed for evaluation by a reviewer.

Other types of quantitative boundary characteristics 36 may also be calculated. For example, a quantitative descriptor of the variation of brightness of the tumor boundary 32 (e.g., the standard deviation of the pixel intensities for those pixels corresponding to the tumor boundary 32) may also be calculated. In addition, in some embodiments the quantitative boundary characteristics 36 may include the number of discontinuities or breaks 54 in the tumor boundary 32, as well as, the length of each discontinuity 54. For example, the length of each discontinuity 54 may be described by equation (1) as follows:

$${L}_{disc} = \frac{arc length of the discontinuity * 100}{360} \% \qquad ( 1 )$$

where L_{disc}, refers to the length of the discontinuity.

A further descriptor which may be quantified in certain embodiments is the squared average contrast. The squared average contrast may be described by equation (2) as follows:

$$C = {( \frac{{I}_{margin}}{{I}_{background}} )}^{2} \qquad ( 2 )$$

where C refers to the squared average contrast, I_{margin} refers to the average pixel intensity in the tumor boundary 32, and I_{background} refers to the average pixel intensity in the background region surrounding the tumor boundary 32. In the depicted embodiment, the thickness of the background region used in quantifying and generating characteristics 36 such as the squared average contrast may be adjusted by the operator, such as via slider 58 of the user interface screen. Adjusting the amount or thickness of the region designated as background may vary the sensitivity and/or accuracy of the generated quantitative boundary characteristics 36. In implementations where different dyes are ranked with respect to each other, it may be useful to keep the thickness of background region constant. In one embodiment, the background region thickness is set to a default of forty-one pixels.

Yet another boundary characteristic 36 that may be quantified in certain embodiments may be rotational contrast, i.e., the ratio of the rotational average of the tumor boundary pixel intensity to the rotational average of the background pixel intensities surrounding the tumor boundary 32. In such an embodiment, the rotational average may be considered the average of the average brightness along the radius around 360 degrees. The rotational contrast may be described by equation (3) as follows:

$${C}_{rotational} = {( \frac{{I}_{rot\_margin}}{{I}_{rot\_background}} )}^{2} \qquad ( 3 )$$

Wherein Cᵣₒₜₐₜᵢₒₙₐₗ refers to the rotational contrast, Iᵣₒₜ_{_{—}}_{margin} refers to the rotational average pixel intensity of the tumor boundary 32, and Iᵣₒₜ_{_{—}}_{background} refers to the rotational average pixel intensity of the background region surrounding the tumor boundary 32. Thus, in one such embodiment where rotational contrast is calculated, the tumor is modeled as a circular region and the highlighted region, i.e., the automatically identified boundary, is considered. In such an embodiment, higher values may be awarded to those dyes that partially illuminate the tumor, i.e., which are limited to the boundary region without highlighting the tumor interior. As will be appreciated, some or all of these quantitative descriptors, and/or different combinations of these descriptors, may be employed in different embodiments.

With the foregoing in mind, it should be appreciated that quantitative boundary characteristics 36 may be generated in a variety of contexts for different dyes, tumor types, points in time, lab animal types, and so forth. These quantitative descriptors may be used to select or grade dyes based on their suitability in different clinical contexts or to select dyes for further testing.

For example, in one embodiment, an operator may process a plurality of images as described herein. In such an embodiment, the operator may access (block 20) a plurality of images 22, such as IR images, of tumors suitably stained with one or more fluorescent or other suitable dyes. The operator may exclude (block 24) those images which exhibit poor or unsuitable staining characteristics from further consideration. In one embodiment, the operator may process the remaining images to select (block 26) the respective tumors 28 within each image 22. One or more automated routines may be executed to identify (block 30) the boundaries of each selected tumor 28. As will be appreciated, the identification of tumor boundaries may occur in a batch processing of the images 22 or may be performed on each image 22 separately as the tumor 28 is selected. The identification of tumor boundaries may be performed contemporaneous with or subsequent to the execution of other routines to enhance the tumor boundaries, such as routines for implementing one or more anisotropic smoothing operations, contrast stretching, multi-stage binarization, and so forth.

One or more automated routines may be implemented to determine (block 34) characteristics 36, such as quantitative measures, of each tumor boundary 32. In certain embodiments, the quantitative descriptors may be standardized (block 80) or normalized for each tumor boundary 32. For example, such standardization processes may account for variations in brightness and/or other image property differences. In one such embodiment, the operator may select a dark area in the respective image 22. The routine calculating the boundary characteristics 36 may in turn use the intensity of the selected dark region (or an average of the intensity in the selected dark region) to normalize or otherwise adjust for differences in brightness between images 22. In this way, differences in image brightness may be normalized by establishing a base darkness level for each image which may be used to scale other intensity levels in the respective image 22.

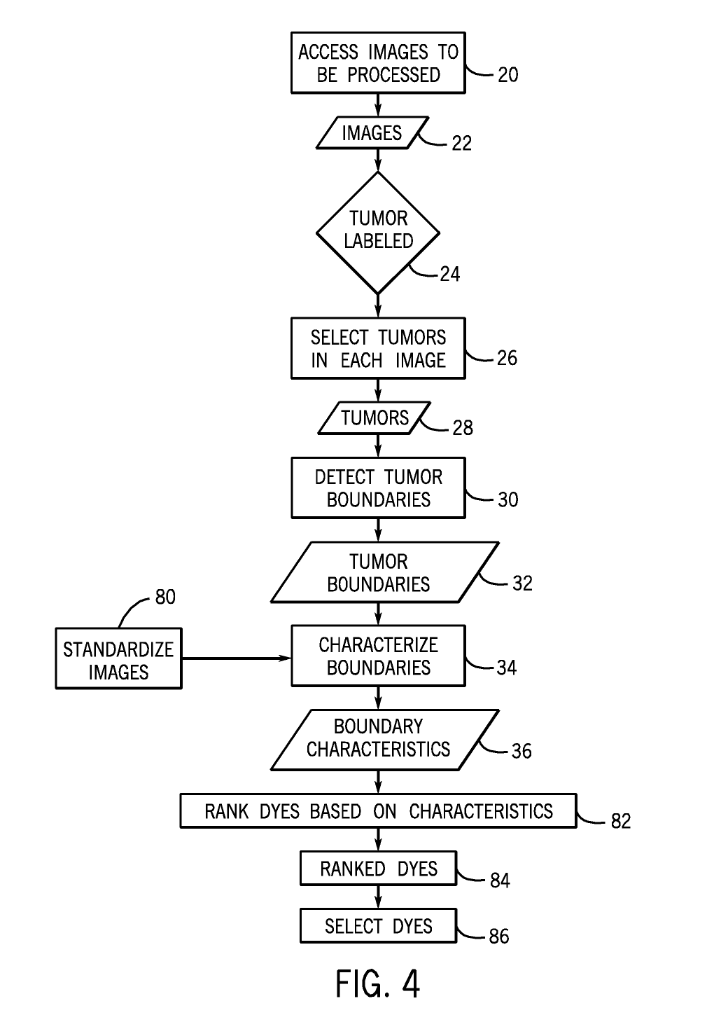

In this manner, comparable quantitative boundary characteristics 36 may be generated for the respective tumor boundaries 32 observed in each processed image 22. The boundary characteristics 36 may then be ranked (block 82), either automatically or by a reviewer, by one or more of the characteristics, allowing a reviewer to select (block 86) which dyes 84 performed best in different medical contexts, such as in different animal models, on different tumor types, based on clearance rate, and so forth. Selected dyes may then undergo further testing and/or may be selected for use in invasive procedures, such as in surgical procedures for tumor removal. In this way, a reviewer may select dyes based on quantitative measurements, as opposed to a subjective visual assessment. As will be appreciated, the order in which different steps illustrated in FIG. 4 may vary. For example, the depicted standardization step may be performed prior or subsequent to when depicted.

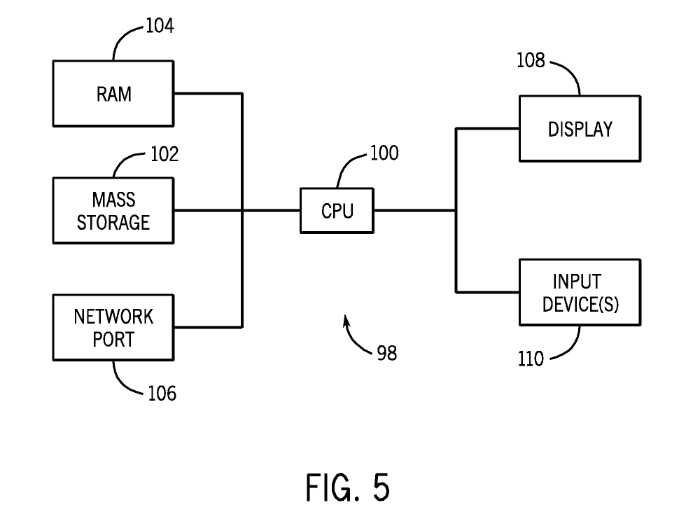

Referring now to FIG. 5, a block diagram depicting a processor-based system 98, such as a computer or workstation, for use in accordance with the present disclosure is provided. The depicted processor-based system 98 includes a microprocessor or CPU 100 capable of executing routines such as those described herein, i.e., routines for tumor boundary detection and computation of quantitative characteristics of such boundaries. Such routines, as well as image data to be processed by such routines and the output (i.e., results) of such routines, may be stored in a local or remote mass storage device 102, such as a hard disk, solid state memory component, optical disk, and so forth. In addition, the processor based system. Further, the processor-based system 98 may access routines or image data for processing via a network connection 106, such as a wired or wireless network connection. Such routines and/or image data may be temporarily stored in RAM 104 prior to processing by the CPU 100.

Accessed or processed image data, as well as the boundary characteristics described herein, may be displayed on a display 108 for review by an operator. In addition, the processor-based system 98 may include one or more input devices 110, such as a keyboard, mouse, touchscreen, touchpad, and so forth, allowing an operator to access image data, select images for processing, select tumors, within images, review results, and so forth. In this manner, an operator may review the outputs of the disclosed techniques and provide inputs to further operation of the disclosed techniques.

The identification of tumor boundaries and quantification of dyes used to highlight the tumor boundaries, as described herein, provides a useful tool to the medical and scientific community. For instance, with the methods outlined above a number of dyes can be analyzed and the data obtained stored to allow comparisons between the dyes to determine the best dyes in general and for specific tumor types. In addition, the efficacy of a dye can be shown over multiple tumor types. Possessing quantitative measurements introduces reliability and reproducibility in assessing the dyes, removing the subjectivity normally involved.

Another benefit of the methods is the automatic detection and marking of the tumor boundary, once the operator selects an area of interest, provides an invaluable tool in a dynamic environment such as a surgical setting. Applying these methods to imaging systems used in open surgery would improve the ability of the surgeon to remove the complete tumor while sparing as much of the normal tissue in the patient as possible.

Technical effects of the invention include the automated or semi-automated identification of tumor boundaries and the quantification of dye efficacy in staining the boundaries. Such measures may allow the analysis and comparison of multiple dyes in a quantitative, objective manner.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method, comprising:
accessing an image of a subject, wherein the subject is administered an agent labeled with a dye prior to generation of the image;
selecting a tumor labeled with the dye from the image;
employing a first routine to detect some or all of the boundary of the tumor; and
employing a second routine to measure one or more characteristics of the boundary.

2. The method of claim 1, comprising reviewing the measurements of the one or more characteristics.

3. The method of claim 1, wherein the first routine enhances the tumor boundary using one or more of an anisotropic filter, contrast stretching, or multi-stage binarization.

4. The method of claim 1, wherein the second routine measures one or more of a squared average contrast, an average intensity, a variance of intensity, a brightness ratio, an average contrast, a rotational contrast, number of discontinuities in the tumor boundary, relative length of each discontinuity in tumor boundary, or a clearance rate.

5. A method of selecting dyes, comprising:
accessing a plurality of images of tumors, wherein the tumors are each stained with a respective image-enhancing dye of a plurality of dyes prior to imaging;
processing the plurality of images to identify the respective tumor boundaries within each image;
employing one or more routines to calculate one or more quantitative characteristics of each tumor boundary; and
selecting one or more of the plurality of dyes based on the one or more quantitative characteristics.

6. The method of claim 5, wherein selecting one or more of the plurality of dyes comprises ranking the dyes based on the quantitative characteristics of each tumor boundary.

7. The method of claim 5, wherein selecting one or more of the plurality of dyes comprises selecting a dye based on one or more of a squared average contrast, an average intensity, a variance of intensity, a brightness ratio, an average contrast, a rotational contrast, number of discontinuities in the tumor boundary, relative length of each discontinuity in tumor boundary, or a clearance rate associated with the dye.

8. The method of claim 5, wherein selecting one or more of the plurality of dyes comprises determining which dyes are suitable for imaging a tumor boundary in one or more of a respective animal model, a respective tumor type, or at a respective clearance rate.

9. The method of claim 5, wherein processing the plurality of images comprises utilizing a computer-executed algorithm to identify tumor boundaries.

10. The method of claim 9, wherein the computer-executed algorithm accepts respective user input indicating the location of a tumor in each respective image prior to identifying the respective tumor boundaries.

11. The method of claim 5, wherein the one or more routines are executed on a processor based system.

12. A method for processing infrared image data to identify a tumor's boundary, comprising:
administering an agent labeled with a fluorescent dye to a subject;
generating an infrared image of the subject;
selecting a tumor from the image;
executing a first computer-implemented algorithm to identify the tumor's boundary;
executing a second computer-implemented algorithm to generate one or more quantitative characteristics of the tumor boundary; and
reviewing the one or more quantitative characteristics to assess the performance of the fluorescent dye.

13. The method of claim 12, wherein reviewing the one or more quantitative characteristics comprises:
comparing the one or more quantitative characteristics of the tumor's boundary to corresponding quantitative characteristics generated for other tumor boundaries; and
ranking the fluorescent dye based on the comparison.

14. The method of claim 12, wherein the first computer-implemented algorithm enhances the identified tumor's boundary using one or more of pre-processing filters, contrast stretching, multi-stage binarization, or a combination thereof.

15. The method of claim 12, wherein the one or more quantitative characteristics comprise one or more of a squared average contrast, an average intensity, a variance of intensity, a brightness ratio, an average contrast, a rotational contrast, number of discontinuities in the tumor boundary, relative length of each discontinuity in tumor boundary, or a clearance rate.

16. A method, comprising:
receiving an input indicative of the location of a dye-enhanced tumor in an image;
executing a first routine configured to determine the boundary of the tumor in the image;
executing a second routine configured to calculate one or more quantitative characteristics of the boundary of the tumor; and
storing or displaying the one or more quantitative characteristics.

17. The method of claim 16, wherein the first routine and the second routine are executed on a processor-based system.

18. The method of claim 16, wherein the first routine employs one or more of a pre-processing filter, contrast stretching, multi-stage binarization, or a combination thereof, to enhance the boundary of the tumor.

19. The method of claim 16, wherein the second routine calculates one or more of a squared average contrast, an average intensity, a variance of intensity, a brightness ratio, an average contrast, a rotational contrast, number of discontinuities in the tumor boundary, relative length of each discontinuity in tumor boundary, or a clearance rate.

20. A system, comprising:
a display capable of displaying an image of a dye-enhanced tumor;
an input device configured to receive an operator input indicative of the location of the dye-enhanced tumor in the image;
a storage or memory device storing routines for determine the boundary of the dye-enhanced tumor and for calculating one or more quantitative characteristics of the boundary; and
a processor configured to receiving the operator input, to execute the routines stored in the storage or memory device in view of the operator input, and to display the one or more quantitative characteristics on the display.

21. The system of claim 20, wherein the storage or memory device comprises one or more of RAM, a hard disk, a solid state memory component, or an optical disk.
